(19) United States
(12) Patent Application Publication
(10) Pub. No.: US 2025/0295531 A1
(43) Pub. Date: Sep. 25, 2025

(54) WASHABLE REUSABLE DIAPER

(71) Applicant: GROUPE SOURIS MINI INC., Québec (CA)

(72) Inventor: Annie BELLAVANCE, Québec (CA)

(21) Appl. No.: 18/678,096

(22) Filed: May 30, 2024

(30) Foreign Application Priority Data
Mar. 25, 2024 (CA) 3232852

(51) Int. Cl. A61F 13/49; A61F 13/505; A61F 13/62

(52) U.S. Cl. CPC A61F 13/49003; A61F 13/49017; A61F 13/505; A61F 13/622

(57) ABSTRACT

A diaper comprising front end and rear end portions and a middle portion therebetween. The middle portion comprises an inner base and a pocket structure comprising a pair of space apart of lateral sheets covering a portion of the inner base and rear skirt extending forwardly covering a portion of the inner base. The lateral sheets and the skirt providing for receiving an insert therein and exposing a portion of the insert to be in contact with the body of a baby when the diaper is worn thereby and extending from the urethra and anal areas of the body of the baby to receive and absorb discharge therefrom.

### CROSS-REFERENCE TO RELATED APPLICATIONS

The present application claims priority on Canadian Patent Application Number 3,232,852 filed on Mar. 25, 2024, and incorporated herein by reference in its entirety.

### TECHNICAL FIELD

The present disclosure generally relates to diapers. More particularly but not exclusively, the present disclosure relates to a washable reusable diaper.

### BACKGROUND

Washable and reusable diapers are made of cloth. Conventional washable diapers include a closeable inner pocket for installing an insert therein. The insert is used for absorbing urine and feces. Closed pockets require unbuttoning the pocket, removing the used insert, or buttoning the pocket up when inserting a fresh insert. Indeed, the foregoing requires a lot of preparation time when removing or inserting an insert. Of course, the inserts need to be removed before being washed which some users may find undesirable. The inserts are usually thick which can hinder a baby's movement. Conventional washable diapers are adjusted with snap buttons, which does not allow for being fitted on the baby's body. Moreover, buttons create discomfort for the baby.

### OBJECTS

An object of the present disclosure is to provide a washable reusable diaper

An object of the present disclosure is to provide a washable reusable diaper with a removable and washable insert.

### SUMMARY

In accordance with an aspect of the present disclosure, there is provided a diaper comprising: a main body defining outer and inner sides thereof, opposite lateral sides thereof, front end and a rear end portions thereof and a middle portion interposed between the front end and rear end portions, the middle portion comprising an inner base and a pocket structure comprising a pair of sheets at each of the opposite lateral sides extending from a rear part to a front part of the middle portion and defining respective and adjacent spaced apart side free longitudinal edges thereof, and a skirt extending forwardly from a rear end of the middle portion and covering a portion of the spaced apart sheets and defining a rear free longitudinal edge, the spaced apart side free longitudinal edges and the rear free longitudinal edge exposing a portion of the inner base therebetween; and a longitudinal absorptive insert for being fitted in the diaper by being positioned on the inner base beneath the pair of sheets and the skirt, the spaced apart side free longitudinal edges and the rear free longitudinal edge providing for an exposed insert portion therebetween with a remainder of the insert portion being covered by the pair of sheets and the skirt, wherein the front end portion and the rear end portions are connectable at the opposite lateral sides to define a closed position of the diaper forming a pair of leg openings at each of the opposite lateral sides and a top waist opening and wherein the insert is fitted in the diaper in the closed position, the exposed portion of the inert is in contact with urethra and anal areas of a baby body to receive and absorb discharge therefrom.

In an embodiment, the insert comprises a thickness between about 60 mm to 70 mm.

In an embodiment, A the absorption capacity of the insert is between about 6 oz to about 10 oz.

In an embodiment, the insert comprises multiple layers stitched together at a common peripheral edge, the multiple layers comprising a top first layer made of a soft absorbent material for being in contact with the body of the baby, a second layer positioned beneath the first layer and comprising absorbent material, a third layer comprising sub-payers made of bamboo and a fourth bottom layer made of carbon.

In an embodiment, the lateral sides of the main body comprise respective main body inwardly curved sections. In an embodiment, the insert defines later sides thereof with respective insert inwardly curved sections corresponding to the main body inwardly curved sections. In an embodiment, the main body inwardly sections define elasticized free edges, each free edge defining a respective inner hem when the diaper is in the closed position inwardly of a respective one of the leg openings for being snugly fitted on a respective leg of the baby body. In an embodiment, the inwardly curve sections are elasticized.

In an embodiment, the rear position comprises an elasticized waste band extending rearwardly from the rear part of the of the middle portion to fittingly engage the back of the baby body when in the closed position.

In an embodiment, the diaper is devoid of any buttons. In an embodiment, the pocket structure is devoid of any buttons.

In an embodiment, the rear end part comprises laterally extending arms extending along the lateral sides when the diaper is in the closed position and defining inner sided thereof for mutually mating with outer sides of the front end portion via hook and loop attachments. In an embodiment, the laterally extending arms are foldable and self-attachable at the inner sides thereof via the hook and loop attachments when in the diaper is in the open position.

In an embodiment, the skirt comprises excess material to form a supple and wavy piece of material that is easily openable or movable so as to receive fecal discharge thereunder.

In an embodiment, the skirt is about between ⅕ to ⅙ of the length of the base sheet.

In an embodiment, the pair of sheets comprise impermeable material to redirect urine discharge towards the insert and away from the body of the baby.

In an embodiment, the pair of sheets cover between about 30% to 40% of the width of the base sheet.

In embodiment, the diaper comprises front end and rear end portions and a middle portion therebetween. The middle portion comprises an inner base and a pocket structure comprising a pair of space apart of lateral sheets covering a portion of the inner base and rear skirt extending forwardly covering a portion of the inner base. The lateral sheets and the skirt providing for receiving an insert therein and exposing a portion of the insert to be in contact with the body of a baby when the diaper is worn thereby and extending from the urethra and anal areas of the body of the baby to receive and absorb discharge therefrom.

Other objects, advantages and features of the present disclosure will become more apparent upon reading of the following non-restrictive description of illustrative embodiments thereof, given by way of example only with reference to the accompanying drawings.

### BRIEF DESCRIPTION OF THE DRAWINGS

The following drawings form part of the present specification and present disclosure. In the appended drawings:

FIG. 1 is a front view of the washable reusable diaper in closed position in accordance with a non-restrictive illustrative embodiment of the present disclosure;

FIG. 2 is a rear view of the washable reusable diaper of FIG. 1 in accordance with a non-restrictive illustrative embodiment of the present disclosure;

FIG. 3 is a side view of the washable reusable diaper of FIG. 1 in accordance with a non-restrictive illustrative embodiment of the present disclosure;

FIG. 4 is a top view the washable reusable diaper of FIG. 1 in accordance with a non-restrictive illustrative embodiment of the present disclosure;

FIG. 5 is a front view of the washable reusable diaper of FIG. 1 in an open position in accordance with a non-restrictive illustrative embodiment of the present disclosure;

FIG. 6 is a rear view of the washable reusable diaper of FIG. 5 in accordance with a non-restrictive illustrative embodiment of the present disclosure;

FIG. 7 is a front view of the washable reusable diaper of FIG. 5 with the insert thereof having been removed in accordance with a non-restrictive illustrative embodiment of the present disclosure;

FIG. 8 is a front view of an insert of the washable reusable diaper of FIGS. 1 to 7 in accordance with a non-restrictive illustrative embodiment of the present disclosure;

FIG. 9 is a rear view of an in an insert of the washable reusable diaper of FIGS. 1 to 7 in accordance with another non-restrictive illustrative embodiment of the present disclosure;

FIG. 10 is a schematic representation of an exploded view of the insert of FIG. 8 or 9 in accordance with a non-restrictive illustrative embodiment of the present disclosure; and

FIG. 11 shows a table of an insert absorption test (ISO 11948-1) for three inserts, A, B and C of differing lengths and a measurement guide for the dimensions of the three inserts in accordance with a non-restrictive illustrative embodiment of the present disclosure.

### DETAILED DESCRIPTION

Generally stated and in accordance with an aspect of the present disclosure, there is provided a diaper comprising a main body defining outer and inner sides thereof, opposite lateral sides thereof, front end and a rear end portions thereof and a middle portion interposed between the front end and rear end portions. The middle portion comprises an inner base and a pocket structure comprising a pair of sheets at each of the opposite lateral sides extending from a rear part to a front part of the middle portion and defining respective and adjacent spaced apart side free longitudinal edges thereof, and a skirt extending forwardly from a rear end of the middle portion and covering a portion of the spaced apart sheets and defining a rear free longitudinal edge. The spaced apart side free longitudinal edges and the rear free longitudinal edge expose a portion of the inner base therebetween. A longitudinal absorptive insert for being fitted in the diaper by being positioned on the inner base beneath the pair of sheets and the skirt. The spaced apart side free longitudinal edges and the rear free longitudinal edge provides for an exposed insert portion therebetween with a remainder of the insert portion being covered by the pair of sheets and the skirt. The front end portion and the rear end portions are connectable at the opposite lateral sides to define a closed position of the diaper forming a pair of leg openings at each of the opposite lateral sides and a top waist opening and wherein the insert is fitted in the diaper in the closed position. The exposed portion of the inert is in contact with urethra and anal areas of a baby body to receive and absorb discharge therefrom.

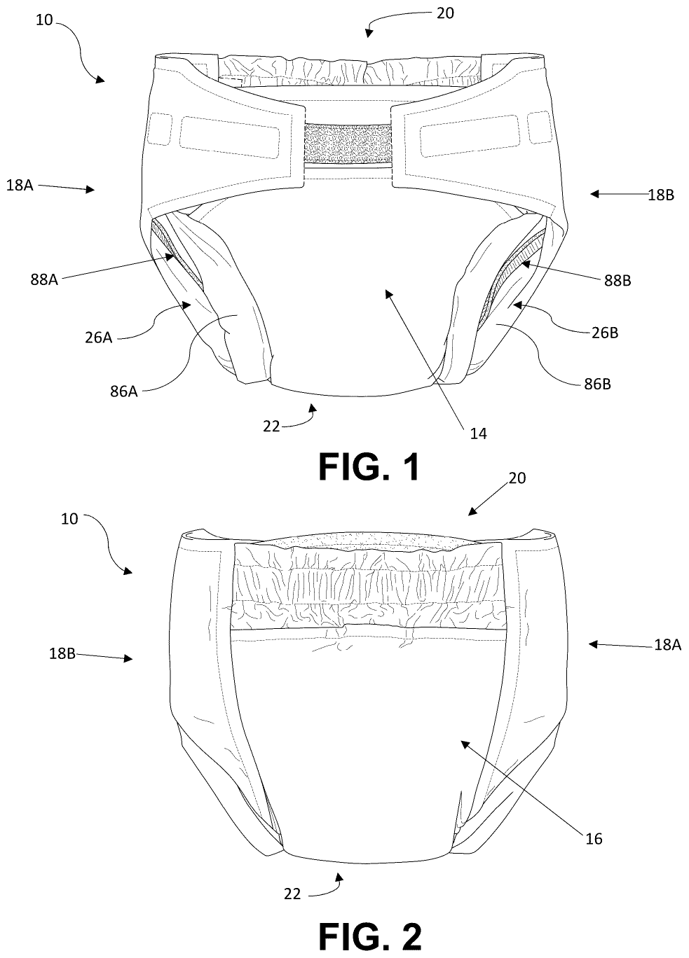

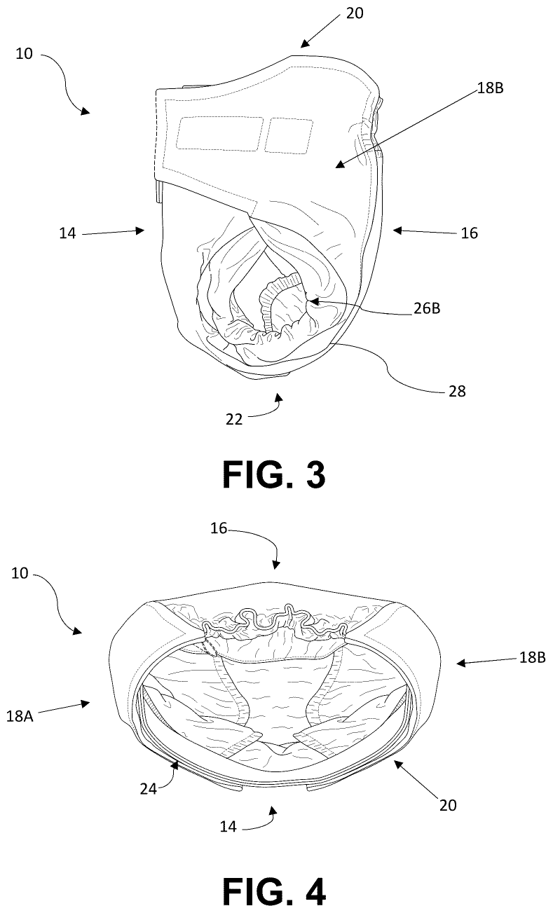

With reference to FIGS. 1 to 11 the washable and reusable diaper and insert thereof of the present disclosure will be described by way of non-restrictive illustrative embodiments.

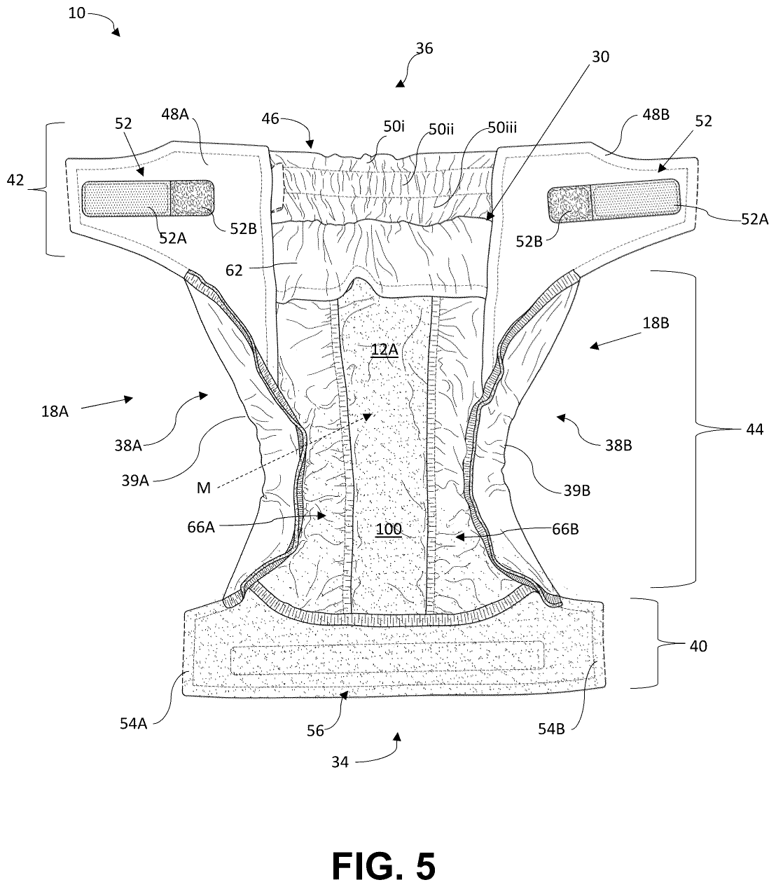

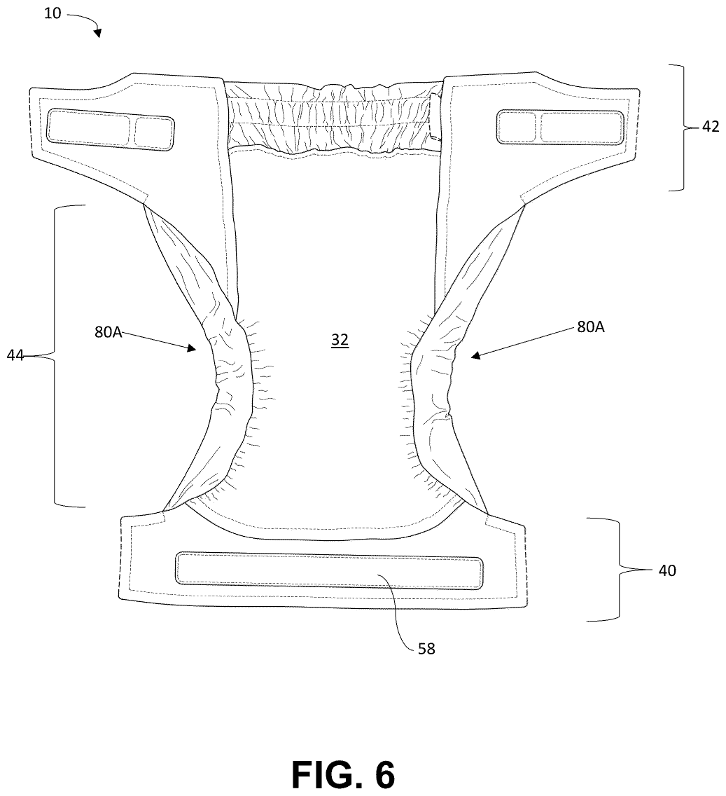

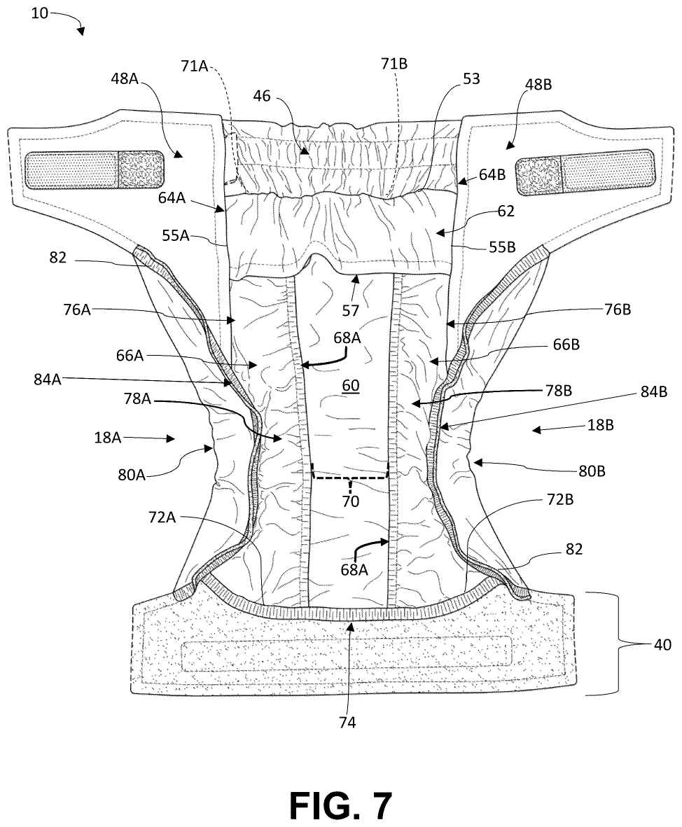

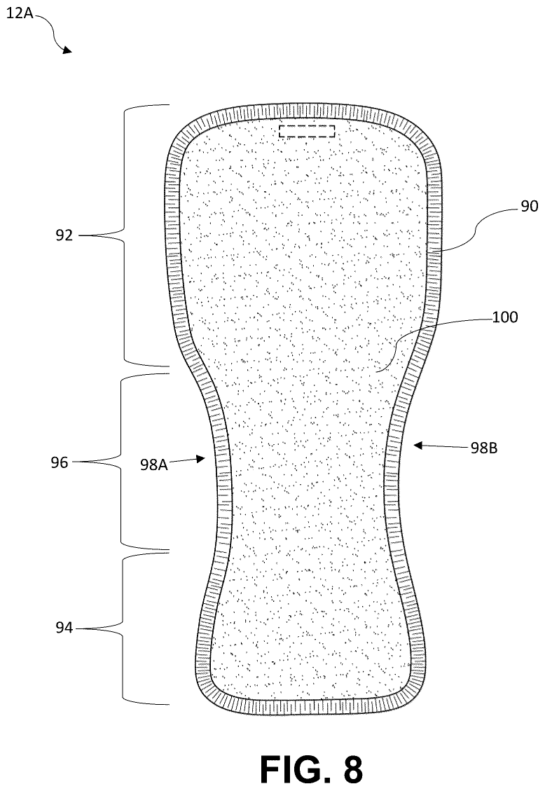

FIGS. 1 to 4 show a diaper 10 in a closed position and FIGS. 5 to 6 show the diaper in an open position. FIG. 7 shows the diaper in an open position without the insert. The inserts 12A, 12B and 12 are respectively shown in FIGS. 8, 9 and 10.

The diaper 10 when in a closed position as shown in FIGS. 1 to 4 defines front and rear faces, 14 and 16, lateral sides 18A and 18B and top and bottom ends 20 and 22. The diaper 10 forms a waist opening 24 at its top end 20 and lateral leg openings 26A and 26B at lateral sides 18A and 18B respectively. The diaper forms a C-shaped shell body 28 spanning the front and rear sides 14 and 16 and bottom end 22 therebetween.

With reference to Figured 5 and 6, the diaper 10 when in an open position defines an inner side 30 and an outer side 32, front and rear ends, 34 and 36, that together define the top end 20 when in the closed position and lateral sides 38A and 38B. The front and rear ends 34 and 36 define the length of the diaper therebetween.

More specifically, the diaper 10 includes a front part 40, a rear part 42 and a median part 44 therebetween 44. The lateral sides 38A and 38B of the diaper define C-shaped inward curved edges 39A and 39B running from the front part 40 along the median part 44 to the rear part 42 thereby the diaper 10 has a variable width W along the length L wherein the width progressively decreases from the rear end 36 towards the middle M of the diaper 10 and then progressively increases from the middle M towards the front end 34.

The rear part 42 includes a waist band 46 interposed between a pair of extending arms 48A and 48B. The waist band 46 includes three sections 50i, 50ii and 50iii, each being formed of a soft tunneled fabric with a respective elastic therein. Accordingly, the waist band 46 provides for a fitted and comfortable fit on the waist and back avoiding fecal leaks.

The extending arms 48A and 48B includes fasteners 52 on the inner side 30 such as hook-and-loop fasteners. In an embodiment, the fastener has mutually fastening parts 52A and 52B for folding each arm 48A and 48B on itself which is convenient during folding and washing for example.

The front part 40 includes a long band defining lateral ends 54A and 54B and middle portion 56 therebetween having a front fastener 58 on the outer side 32 for being connected to the fasteners 52 when the diaper 10 is in the closed position.

Turning to FIG. 7, the diaper 10 includes a base sheet 60 with additional material sewed thereon to provide barriers and pockets as will be further described.

A skirt 62 forward the waist band 46 is interposed between the extending arms 48A and 48B and defines a rear end 53 and lateral sides 55A and 55B which are sewed to the inner base sheet 60 and together with the front free edge 57 form a pocket. The skirt 62 has excess material i.e. more than what is required simply extend between lateral sides 55A and 55B thus forming a supple and wavy piece that is easily openable or movable so as to receive fecal discharge in this rear pocket. The skirt 62 has a length of about ⅓ of the inner borders 64A and 64B of the extending arms 48A and 48B making the pocket deep enough for receiving fecal matter. In an embodiment, the skirt 62 comprises micro-fibers. The skirt 62 is about ⅙ to about ⅕ of the length of the base sheet 60 providing required depth for fecal discharge receipt so as to avoid fecal matter from engaging the body of the baby.

The diaper 10 includes a pair of spaced apart longitudinal sheets 66A and 66B running along the length of the inner base 60 and defining respective free edges 68A and 68B that form a space 70 exposing the base 60 (see FIG. 7) or the insert 12 (see FIG. 5) as will be discussed herein. Each sheet 66A, 66B has a respective rear end 71A, 71B sewn along the rear end 53 and front ends 72A and 72B sewn along a front curved border 74 delimiting the front part 40 and the middle part 44. The lateral sides 76A and 76B run along each lateral side 18A and 18B of the diaper and are sewn to the base sheet 60. Thus, sheets 66A and 66B define along with the base sheet 60 a pair of spaced apart and adjacent pockets 78A and 78B for receiving the insert 12 therein. The insert 12 is fitted into both pockets 78A and 78B via the opening or space 70 and the open edges 68A and 68B. The inner open edges 68A and 68B include elastics to maintain the insert 12 in place within the pockets 78A and 78B.

In an embodiment, the sheets 66A and 66B are made of impermeable material to redirect urine discharge towards the insert and away from the baby's skin. In an embodiment, the sheets 66A and 66B cover between about 30% to 40% of the width of base sheet 60, specifically each sheet 66A and 66B extends inwardly respectively covering 15% to about 20% of a respective lateral end of the base sheet 60 along its width hence the space 70 exposes between about 60% to about 70% of the width of the base sheet 60.

The skirt 62 overlies the sheets 66A and 66B and the space 70 therebetween as well as the exposed insert 12 to form a pocket therewith.

The middle part 44 of the diaper 10 includes curved sides 80A and 80B which comprise a respective fabric with an elastic therein as well as an elastic 82 at their free edges 84A and 84B. In this way, when the diaper 10 is in a closed position a pair of leg fittings 86A and 86B are formed defining the openings 26A and 26B, the material of the leg fittings 86A and 86B is elasticized as well at each inner hem 88A or 88B (see FIG. 1) defined by the free edges 84A and 84B and circumscribing the openings 26A and 26B thereby fittingly grasping each leg of the baby in order to avoid leaks from the diaper 10.

Turning now to FIG. 8 there is shown an insert 12A for being fitted within pockets 78A and 78B defining a peripheral edge 90. The insert 12A is a longitudinal body with a rearward part 92, a forward part 94 and a middle part 96. The rear part 92 is larger than the front part 94 and the middle part is inwardly curved at both lateral edge portions 98A and 98B thereof to corresponds to the inward curved lateral sides 80A and 80B of the diaper 10.

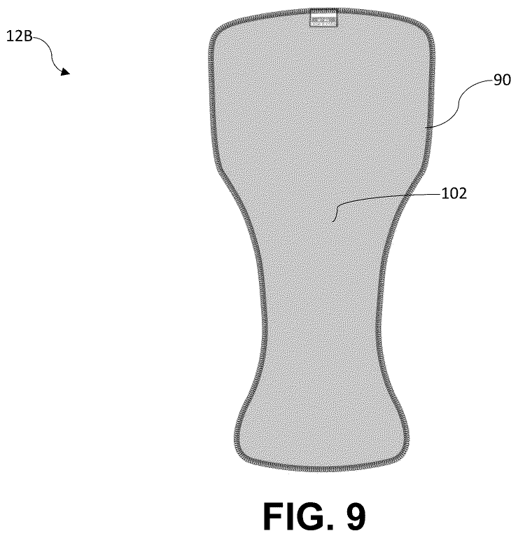

Turning to FIG. 9, there is shown an insert 12B having a similar configuration as insert 12A and thus needs not be further described herein for concision purposes only.

FIG. 8 shows the top surface 100 of the insert 12A and FIG. 9 shows the undersurface 102 of the insert 12B.

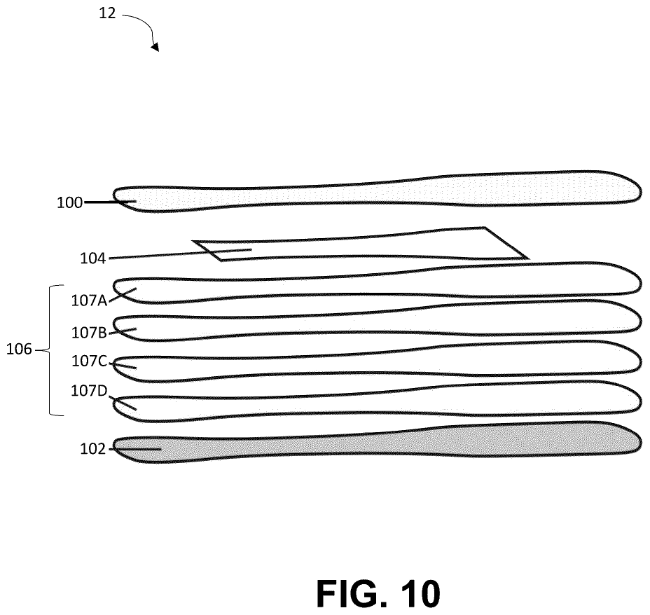

Turning to FIG. 10, an insert 12 as provided herein comprises multiple layers of sheets that are stitched together about the peripheral edge 90.

The top outer layer 100 defines the top surface and comprises a soft absorbent material. In an embodiment, the material of layer 100 is made of micro-suede. In an embodiment, the material of layer 100 is made of about 80% polyester and 20% of polyamide. In an embodiment, the grams per square meter (GSM) density or weight of the material of layer 100 is about 300 gm/m2.

The second layer sheet 104 is positioned beneath the top sheet 100 and reinforces absorption of the outer surface sheet 100 along a length thereof from at least before the area of the outer surface being in contact with the urethra or the urethra region of the baby body to the at least after the area of the outer surface being in contact with the anus or the anal area of the baby body. In an embodiment, this covers about 60% to 90% or 65% to 85% of the length of the insert. In an embodiment, this covers from about 70% to about 80% of the length of the insert. In an embodiment, this covers about 75% of the length of the insert. Indeed, having a shortened length under sheet provides for avoiding additional unnecessary material which increases the thickness of the inserts. In an embodiment, the under sheet is made of micro-suede. In an embodiment, the under sheet is made of microfibers. In an embodiment, the GSM weight of the under sheet 104 is about 330 gm/m2.

The third layer 106 comprises multilayered sheets 107A, 107B, 107C and 107D. In an embodiment, the sheets of the third layer are made of bamboo fabric. In an embodiment, there are 2-5 sheets. In an embodiment, there are 3-4 sheets. In an embodiment, there are 4 sheets as shown in the present non-limiting example. In an embodiment, the third layer 106 is made of 80% viscous bamboo and 20% polyester. In an embodiment, the GSM weight of the third layer 106 is about 250 gm/m2.

The bottom outer sheet 102 defines the undersurface or bottom surface. In an embodiment, the bottom layer sheet 102 is made of carbon fiber to reduce odors. In embodiment, sheet 102 is made of about 95% polyester and 5% bamboo carbon. In an embodiment, the GSM weight of the sheet 102 is about 175 gm/m2.

In an embodiment, the thickness of the insert 12 is between about 60 mm to 70 mm. In an embodiment, the thickness of the insert is about 65 mm.

Indeed, a thinner and shorter insert provides an overall thinner diaper 10 with a standard absorption all the while providing greater comfort and movement to the baby. This avoids a thick diaper structure in favor of a more ergonomic design that does not reduce absorption all the while having several barriers described herein that avoid diaper leakage.

In an embodiment, the material used for the diaper 10 comprises elastane which provides for flexibility, and freedom of movement. The lack of buttons in favor of hook-and-loop fasteners provides greater comfort. The diaper requires only one step for assembly and disassembly namely fitting the insert in the pocket and removing it from the pocket. The structure of the diaper and insert provides a more fitted and thinner garment making it more comfortable, more breathable and less cumbersome to movement in addition to making it easier to dress the baby as the diaper does not take up much space.

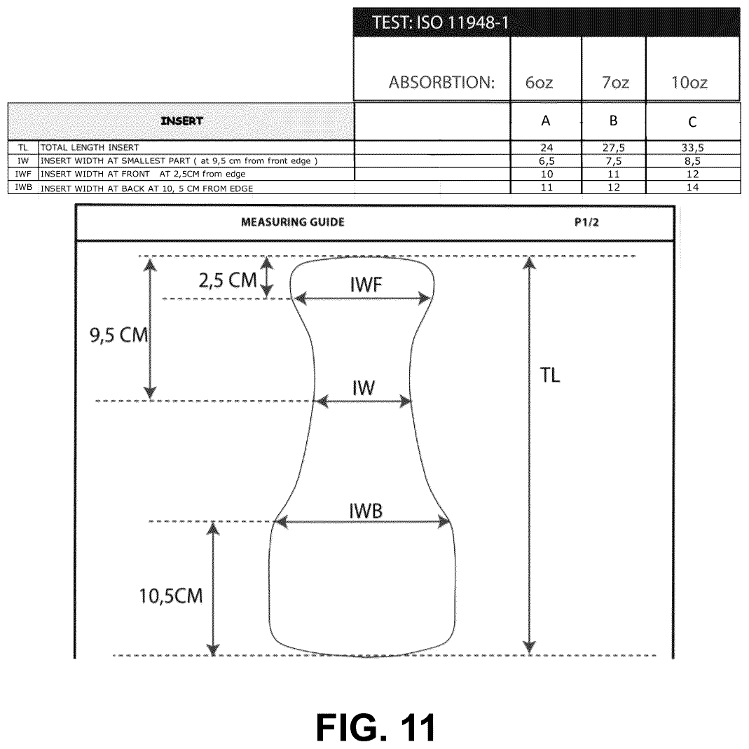

FIG. 11 shows the dimensions of various three inserts (A, B, C) having three respective lengths and respective dimensions and ratios. The total absorption capacity of inserts A, B and C are respectively 6 oz, 7 oz and 10 oz. Thus, the absorption capacity of the insert is about 6 oz to about 10 oz depending on the length. The insert is about 65 mm thick. The open cropping pocket of the diaper avoids thick enclosures thereby providing for a thinner diaper structure which offers greater mobility. Thus, the performance of the diaper and insert are not compromised by the thinner and more fitted structure.

The various features described herein can be combined in a variety of ways within the context of the present disclosure so as to provide still other embodiments. As such, the embodiments are not mutually exclusive. Moreover, the embodiments discussed herein need not include all of the features and elements illustrated and/or described and thus partial combinations of features can also be contemplated. Furthermore, embodiments with less features than those described can also be contemplated.

It is to be understood that the present disclosure is not limited in its application to the details of construction and parts illustrated in the accompanying drawings and described hereinabove. The disclosure is capable of other embodiments and of being practiced in various ways. It is also to be understood that the phraseology or terminology used herein is for the purpose of description and not limitation. Hence, although the present disclosure has been provided hereinabove by way of non-restrictive illustrative embodiments thereof, it can be modified, without departing from the scope, spirit and nature thereof and of the appended claims.

What is claimed is:

1. A diaper comprising:
a main body defining outer and inner sides thereof, opposite lateral sides thereof, front end and a rear end portions thereof and a middle portion interposed between the front end and rear end portions, the middle portion comprising an inner base and a pocket structure comprising a pair of sheets at each of the opposite lateral sides extending from a rear part to a front part of the middle portion and defining respective and adjacent spaced apart side free longitudinal edges thereof, and a skirt extending forwardly from a rear end of the middle portion and covering a portion of the spaced apart sheets and defining a rear free longitudinal edge, the spaced apart side free longitudinal edges and the rear free longitudinal edge exposing a portion of the inner base therebetween; and
a longitudinal absorptive insert for being fitted in the diaper by being positioned on the inner base beneath the pair of sheets and the skirt, the spaced apart side free longitudinal edges and the rear free longitudinal edge providing for an exposed insert portion therebetween with a remainder of the insert portion being covered by the pair of sheets and the skirt,
wherein the front end portion and the rear end portions are connectable at the opposite lateral sides to define a closed position of the diaper forming a pair of leg openings at each of the opposite lateral sides and a top waist opening and wherein the insert is fitted in the diaper in the closed position, the exposed portion of the inert is in contact with urethra and anal areas of a baby body to receive and absorb discharge therefrom.

2. A diaper according to claim 1, wherein the insert comprises a thickness between about 60 mm to 70 mm.

3. A diaper according to claim 1, wherein the absorption capacity of the insert is between about 6 oz to about 10 oz.

4. A diaper according to claim 1, wherein the insert comprises multiple layers stitched together at a common peripheral edge, the multiple layers comprising a top first layer made of a soft absorbent material for being in contact with the body of the baby, a second layer positioned beneath the first layer and comprising absorbent material, a third layer comprising sub-layers made of bamboo and a fourth bottom layer made of carbon.

5. A diaper according to claim 1, wherein the lateral sides of the main body comprise respective main body inwardly curved sections.

6. A diaper according to claim 5, wherein the insert defines later sides thereof with respective insert inwardly curved sections corresponding to the main body inwardly curved sections.

7. A diaper according to claim 5, wherein the main body inwardly sections define elasticized free edges, each free edge defining a respective inner hem when the diaper is in the closed position inwardly of a respective one of the leg openings for being snuggly fitted on a respective leg of the baby body.

8. A diaper according to claim 7, wherein the inwardly curve sections are elasticized.

9. A diaper according to claim 1, wherein the rear position comprises an elasticized waste band extending rearwardly from the rear part of the of the middle portion to fittingly engage the back of the baby body when in the closed position.

10. A diaper according to claim 1, wherein the diaper is devoid of any buttons.

11. A diaper according to claim 1, wherein the pocket structure is devoid of any buttons.

12. A diaper according to claim 1, wherein the rear end part comprises laterally extending arms extending along the lateral sides when the diaper is in the closed position and defining inner sided thereof for mutually mating with outer sides of the front end portion via hook and loop attachments.

13. A diaper according to claim 12, wherein the laterally extending arms are foldable and self-attachable at the inner sides thereof via the hook and loop attachments when in the diaper is in the open position.

14. A diaper according to claim 1, wherein the skirt comprises excess material to form a supple and wavy piece of material that is easily openable or movable so as to receive fecal discharge thereunder.

15. A diaper according to claim 1, wherein the skirt is about between ⅕ to ⅙ of the length of the base sheet.

16. A diaper according to claim 1, wherein pair of sheets comprise impermeable material to redirect urine discharge towards the insert and away from the body of the baby.

17. A diaper according to claim 1, wherein the pair of sheets cover between about 30% to 40% of the width of the base sheet.
